(19) United States
(12) Patent Application Publication
(10) Pub. No.: US 2022/0361801 A1
(43) Pub. Date: Nov. 17, 2022

(54) SYSTEM AND METHOD FOR PROVIDING REAL-TIME BIOLOGICAL FEEDBACK TRAINING THROUGH REMOTE TRANSMISSION

(71) Applicant: ExeBrain Co., Ltd., Taipei City (TW)

(72) Inventors: Shu-Chun Kuan, Taipei City (TW); Jason Chun-Cheng Lin, Taipei City (TW); Chung-Tse Shen, Taipei City (TW)

(21) Appl. No.: 17/495,805

(22) Filed: Oct. 6, 2021

(30) Foreign Application Priority Data
May 14, 2021 (TW) 110117576

(51) Int. Cl. A61B 5/375; A61B 5/024; A61B 5/00; A61B 5/291; A61B 5/372

(52) U.S. Cl. CPC A61B 5/375; A61B 5/02405; A61B 5/0006; A61B 5/291; A61B 5/372; A61B 5/6815; A61B 2560/0456

(57) ABSTRACT

A system for providing real-time biological feedback training through remote transmission is provided and includes a local brain wave collection device, a docking device, and a dongle. The local brain wave collection device is used to detect a brain wave and a heart rate variability data of a subject. The docking device communicates with the local brain wave collection device remotely to connect a remote cloud system to compare the brain wave and the heart rate variability data with a brain wave database to generate a comparison result, and according to the comparison result, the system provides the subject a feedback training interface.

### CROSS-REFERENCE TO RELATED APPLICATION

This application claims priority of Taiwan patent application No. 110117576, filed on May 14, 2021, the content of which are incorporated herein in its entirety by reference.

### BACKGROUND OF THE INVENTION

### 1. Field of the Invention

The present invention relates to a system and method for providing biological feedback training, and particularly to a system and method for providing real-time biological feedback training through remote transmission.

### 2. The Prior Arts

The existing biological feedback training mainly uses wireless devices at the input, for example, to use a pair of electrode pads to compare brain waves changed before and after training for three areas of the parietal lobe, and use a pair of electrode pads to detect sensorimotor rhythm (SMR) influenced by neurophysiological feedback, or collect physiological signals, and upload physiological data to the cloud platform via a wired or wireless transmission module for analysis. The user needs to open the application or related programs to read the physiological device during sleep by retrospect. However, the existing technology usually makes the subject unable to obtain physiological information such as brain wave or heart rate variability immediately, which requires several hours to several days of reading.

At the same time, although the existing smart bed structure health management system also collects physiological signal which is collected when the individual is lying in bed and sleeping. The physiological signals are uploaded to the cloud platform for analysis via wired or wireless transmission modules. It is necessary to open the relevant application to read the physiological device during sleep in retrospective way. The disadvantage is that the physiological signal of the subject cannot be calculated immediately and fed back in real time after transmission.

In addition, although there is a feedback mechanism for Real time fMRI neurofeedback functional magnetic resonance imaging, the magnetic resonance imaging equipment is quite expensive, most of which is installed in medical institutions. It takes more than 30 minutes to collect signals for imaging, the calculation of the feedback mechanism also takes more than 10 minutes, thus it is impossible to achieve remote local configuration and real-time (within 1 minute) analysis feedback.

Therefore, it is necessary to propose an improved method and system that can provide real-time feedback at the remote end, so that the subject can immediately understand their own condition, and the subject can recover the physiological signals through visual or audial feedback.

### SUMMARY OF THE INVENTION

To solve the above problems, the present invention provides a system for providing real-time biological feedback training through remote transmission, which includes a local brain wave collection device, a docking device, and a dongle. The local brain wave collection device detects a brain wave and a heart rate variability data of a subject to generate a biological database. The dongle is used for establishing wireless or wired communication with the local brain wave collection device to receive the biological database. The docking device is electrically connected to the dongle to upload the biological database to a remote cloud system for abnormal brain wave comparison, and receive a feedback result from the remote cloud system, provide the feedback result through a feedback interface to the subject and allow the subject to adjust physiological signals for recovery. The remote cloud system includes a brain wave database, and the remote cloud system compares the biological database according to the brain wave database to generate the feedback result which is visual or aural in real time.

According to an embodiment of the present invention, the biological database is about brain wave data of 19 channels.

According to an embodiment of the present invention, the remote cloud system includes a cloud server which converts 19-channel brain waves into characteristics including vibration, frequency, brain wave location, and brain wave pattern through calculating frequency spectrum.

According to an embodiment of the present invention, the brain wave database includes health norm and clinical norm for comparing the biological database uploaded by the docking device.

According to an embodiment of the present invention, the remote cloud system includes a conversion device and a feedback device, the conversion device compares the biological database according to the brain wave database to generate a comparison result, and the feedback device generates a feedback result from the comparison result according to a training threshold.

According to an embodiment of the present invention, the feedback result based on locations of the 19 channels is used to build an active area of brain by a combination of brain wave patterns to train a specific area of the brain.

According to an embodiment of the present invention, the local brain wave collection device is a brain wave detection cap.

The present invention further provides a method for providing real-time biological feedback training through remote transmission, which includes using a local brain wave collection device 10 to detect a brain wave and a heart rate variability data of a subject to generate a biological database; using a dongle to receive the biological database; and receiving a feedback result from the remote cloud system, providing the feedback result through a feedback interface to the subject and allowing the subject to adjust physiological signals for recovery. The remote cloud system includes a brain wave database, and the remote cloud system compares the biological database according to the brain wave database to generate the feedback result which is visual or aural in real time.

By using the system and method for biological feedback training of the present invention, real-time feedback can be provided remotely, so that the subject can immediately (for example, within 1 minute) understand their own condition, and the subject can adjust the physiological signals through visual or auditory feedback to recover.

### BRIEF DESCRIPTION OF THE DRAWINGS

FIG. 1 illustrates a block diagram of a system for biological feedback training according to an embodiment of the present invention;

FIG. 2 illustrates a flowchart of a method for biological feedback training according to an embodiment of the present invention;

FIG. 3 illustrates a diagram of usage of a system for biological feedback training according to an embodiment of the present invention;

FIG. 4 illustrates a diagram of a system for biological feedback training according to an embodiment of the present invention;

FIG. 5 illustrates a diagram of analysis of brain wave characteristics according to an embodiment of the present invention;

FIG. 6A illustrates a diagram of a local brain wave collection device according to an embodiment of the present invention; and

FIG. 6B illustrates a sectional view of a local brain wave collection device of FIG. 6A.

### DETAILED DESCRIPTION OF THE PREFERRED EMBODIMENT

The present invention provides a system for providing real-time biological feedback training, which uses a local brain wave collection device 10. It should be noted that the brain wave in the present invention means electroencephalography (EEG). In this embodiment, the local brain wave collection device 10 includes a plurality of electrodes for detecting brain waves of a subject, and each of the electrodes has an output unit, a wireless communication unit, a primary amplifier, a storage unit, and an accelerator. Wireless communication can be established between the wireless communication unit can and a dongle 11 to transmit a biological database of the subject. The local brain wave collection device 10 further includes: ear electrodes located at both ears, the ear electrodes will detect a heart rate variability data (HRV), an autonomic nerve balance, and a brain function of the subject, and transmit blood flow and heart rate variability data for analysis of autonomic nervous function. The local brain wave collection device 10 also includes a secondary amplifier which collects and amplifies the brain wave signals. It should be noted that the above-mentioned features of the local brain wave collection device 10 are only for example, and not used to limit the present invention. The local brain wave collection device 10 may be wired or wireless, which can be connected through an adapter or a dongle 11 respectively to allow the data of the local brain wave collection device 1 to be transmitted remotely.

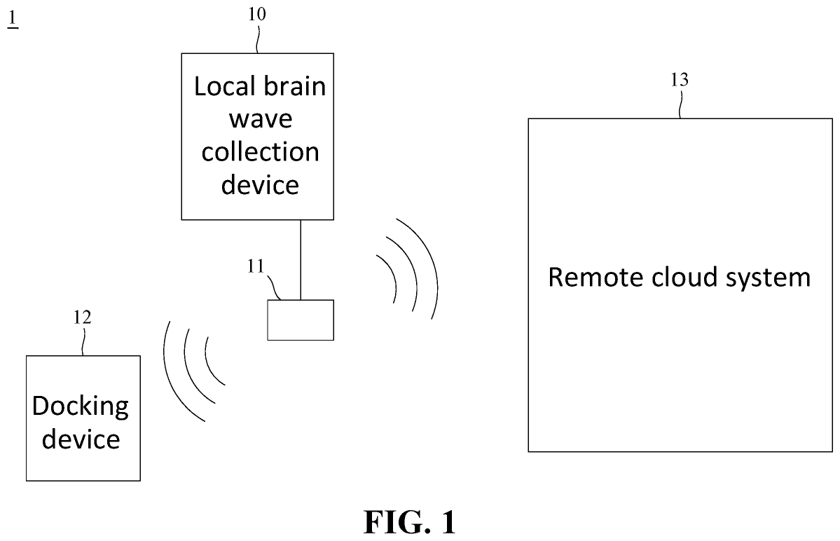

Please refer to FIG. 1, which illustrates a block diagram of a system 1 for biological feedback training according to an embodiment of the present invention. The system 1 for real-time biological feedback training proposed by the present invention uses the local brain wave collection device 10. In an embodiment, the local brain wave collection device 10 is a brain wave detection cap, and the system 1 of the present invention also includes a dongle 11 and a docking device 12, wherein the dongle 11 stores data calculation software. The local brain wave collection device 10 is used to detect a brain wave and a heart rate variability data of a subject to generate a biological database, which is digital data of the brain wave signal and heart rate variability data. The dongle 11 is electrically connected to the docking device 12, which can establish wireless communication with the local brain wave collection device 10 to receive the biological database. The docking device 12, which can be a computer and/or mobile phone, etc., receives the biological database through the dongle 11, and uploads the biological database to a remote cloud system 13 for abnormal brain wave comparison. The docking device 12 also receives a feedback result from the remote cloud system 13, and provides the feedback result through a feedback interface to the subject, let the subject adjust the physiological signals for recovery. The feedback interface is a visual feedback interface or an audial visual feedback interface.

In the embodiment, regarding to the visual or audial feedback results of the present invention, in addition to the surface cortex of the brain, the deep cortex of the brain and specific brain regions can also be trained. In other embodiments, in addition to 19 channels, it can also be a neurophysiological feedback result of 6 channels, 32 channels, 64 channels, etc. In this embodiment, the feedback result based on locations of the 19 channels is used to train a specific area of the brain by a combination of brain wave patterns such as electroencephalogram (EEG).

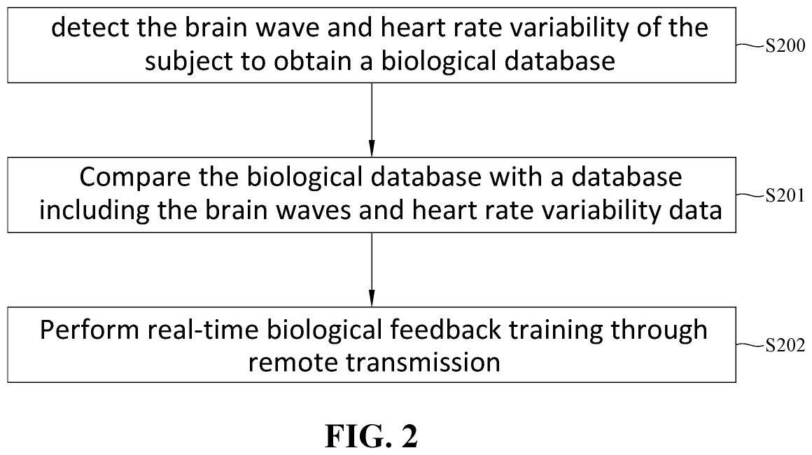

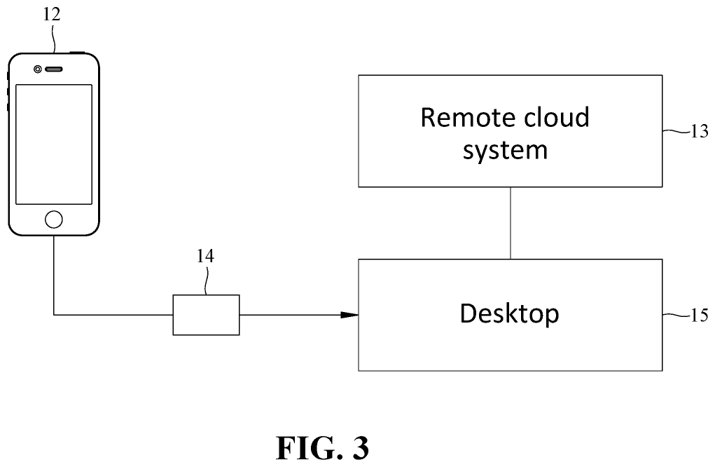

Please refer to FIG. 2, FIG. 2 illustrates a flowchart of a method for biological feedback training according to an embodiment of the present invention. The method for biological feedback training method proposed by the present invention includes three steps: Step S200 is using the local brain wave collection device 10 to detect the brain wave and heart rhythm variability of the subject to obtain a biological database, and upload the biological database to the remote cloud system 13 through the docking device 12; step S201 is that the remote cloud system 13 performs real-time calculations, the biological database is compared with the brain waves of the disease to obtain a comparison result. In this embodiment, the abnormal brain waves of the subject are compared with the normal brain waves. It can be analyzed through 19 locations that whether function of the specific region of the brain waves or neural network is hyper-activity or hypo-activity compared with the norm of database. The purpose is to restore the normal brain wave pattern at the user end through neurophysiological feedback; and step S202 is using the remote cloud system 13 to feedback the comparison result according to a training threshold, that is, a visual or audial feedback result is produced and sent back to the docking device 12 or local device, that is, the desktop 15. The above three steps include immediacy, accuracy and immunity to other noises. In some embodiments, in addition to the computer, the docking device 12 also includes screen synchronization control as a visual feedback interface, and speaker synchronization control as an audial feedback interface, which makes the remote cloud system able to use neural feedback software to adjust the training parameters of the subject. In addition, in an embodiment, when the docking device 12 uses a mobile phone for training, the memory capacity of the mobile phone is limited, as shown in FIG. 3, the mobile phone used as the docking device 12 can use the mobile phone installed with calculation software, which corresponds to an external hard disk device 14, to connect to a local device such as a desktop 15 or a base station, and then the desktop 15 communicates with the remote cloud system 13 through the TCP/IP protocol for remote transmission, so that the subject can use the local screen to receive neurophysiological feedback training through a mobile phone or a neurophysiological feedback set-top box, so that the subject can restore normal brain waves. For example, a subject with inattention can improve concentration through brain training. In the embodiment, in addition to mobile phone, the subject can also receive training through the screen of local display device.

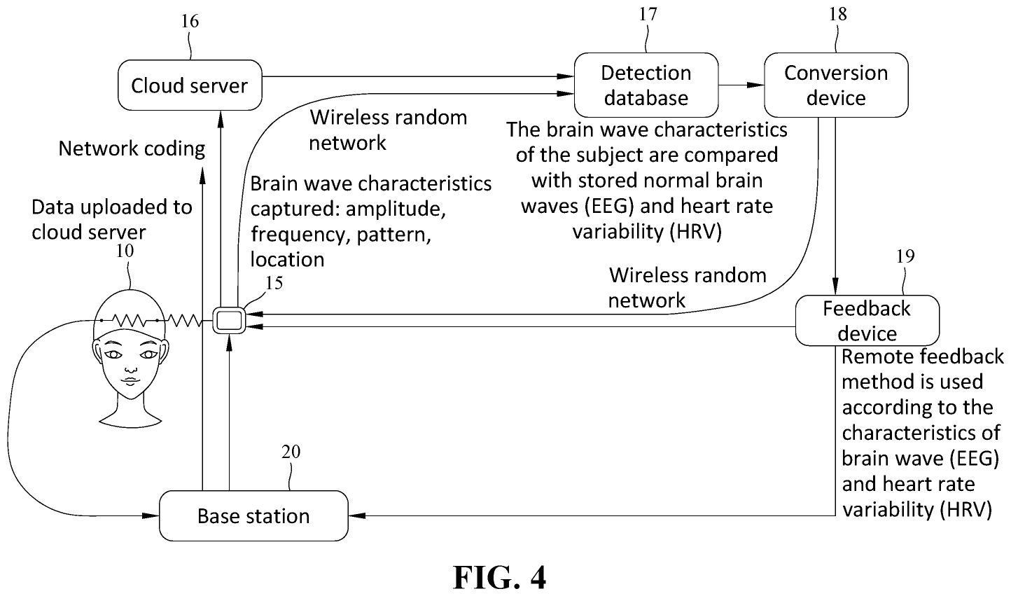

Please refer to FIG. 4, FIG. 4 illustrates a diagram of a system for biological feedback training according to an embodiment of the present invention. As shown in FIG. 4, the local brain wave collection device 10 (the brain wave detection cap here) worn by the subject will be connected to a local device such as desktop 15 and portable computer devices such as laptops, ipads, mobile phones by the above-mentioned dongle 11 for wireless transmission, or the adapter for wired transmission to collect biological database such as brain waves and heart rate variability data.

The local brain wave collection device 10 will also be connected to a base station 20 like a neurophysiological feedback set-top box. The relationship between the local brain wave collection device 10 and the base station 20 of the neurophysiological feedback set-top box is like that between a user host and a game server of an online game. In the embodiment, the brain wave detection device through the set-top box transmits signals remotely, compares the norm, and gives feedback, and the above-mentioned process needs to be completed within 1 second. The base station 20 will also be connected to the desktop 15, and will transmit brain wave characteristics to the cloud server 16 through network coding. The desktop 15 and the cloud server 16 will connect to the brain wave database via a wireless network. That is, the detection database 17 is used to compare the amplitude, frequency, pattern, location and other characteristics of the brain waves. The conversion device 18 will use the stored normal brain waves (Electroencephalography, EEG), heart rate variability (HRV), and the brain wave characteristics of the subject to generate a comparison result in real time, and the feedback device 19 generates a visual or audial feedback result based on a training threshold according to the comparison result, and then transmits the feedback result to the desktop computer 15 via a wireless random network. The feedback device 19 uses a remote feedback method, according to the characteristics of brain wave (EEG) and heart rate variability (HRV), to transmit the real-time feedback result to the desktop 15 and the base station 20, and the feedback result are provided to the subject through the visual or audial feedback interface.

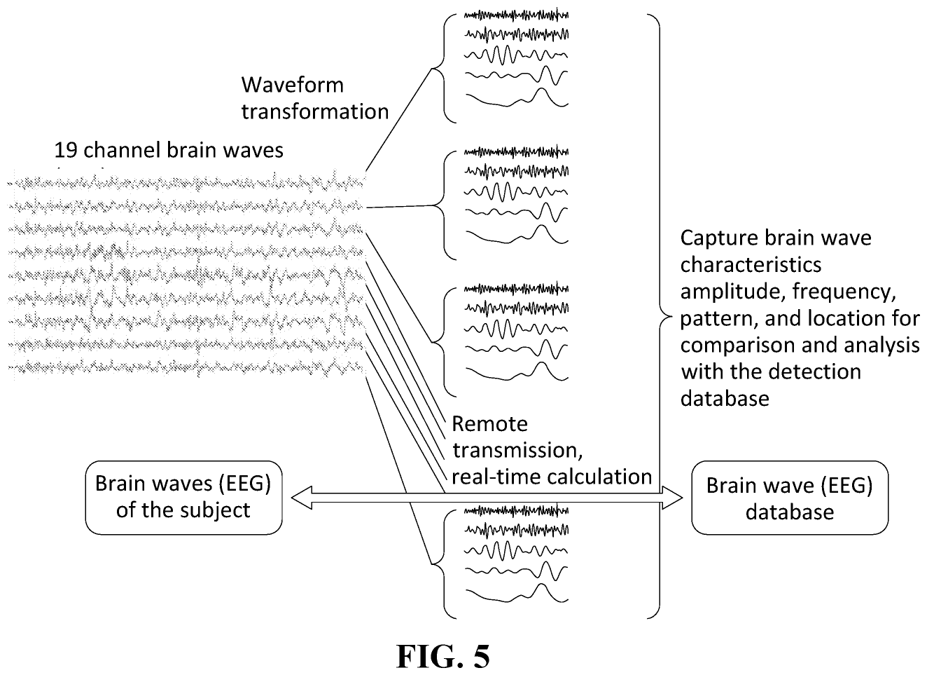

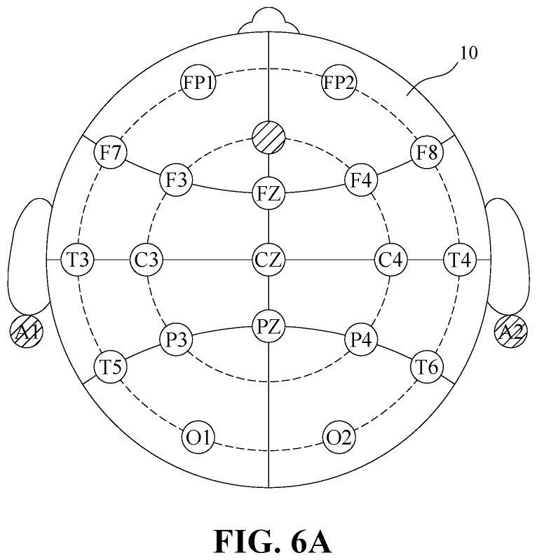

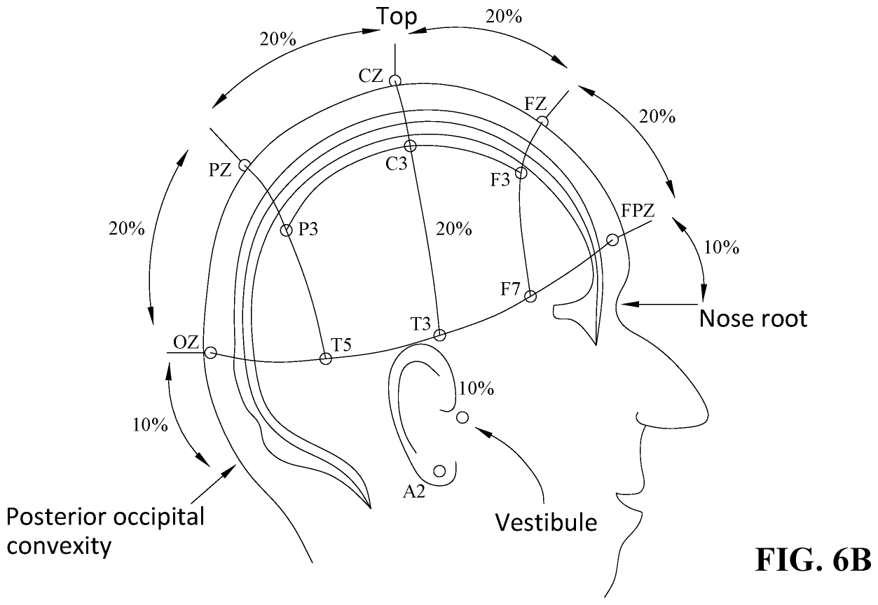

FIG. 5 illustrates a diagram of analysis of brain wave characteristics according to an embodiment of the present invention. As shown in FIG. 5, the brain wave characteristics detected by the 19 channels of the local brain wave collection device 10 (that is, the 19 electrodes FP1, FP2, F3, F4, F7, F8, FZ, T3, C3, CZ, C4, T4, T5, P3, PZ, P4, T6, 01, 02 in FIG. 6) include amplitude, frequency, pattern, location (the location of each of the electrodes FP1, FP2, F3, F4, F7, F8, FZ, T3, C3, CZ, C4, T4, T5, P3, PZ, P4, T6, 01, 02), the amplitude of the brain wave pattern, The four characteristics of frequency, type, and location, and the four characteristic parameters constitute the brain wave database of groups of different ethnics. The active area of brain can be built by the brain wave characteristics based on the locations of the 19 channels to be used as the parameters of the above comparison and training. In this embodiment, the brain waves collected by the local brain wave collection device 10 will obtain a basic score after brain wave pattern comparison, as shown in FIG. 5, after comparison and analysis with the brain wave database, a benchmark point of index scores is generated. This benchmark point is also a difference from the norm in similar ethnic groups (same age, education level, gender, etc.).

For example, if a user's brain waves collected by the local brain wave collection device 10 are converted into a score of X, the user's ideal score should be a score of Y by comparison with the database, then the goal of the neural feedback training is to reduce a difference between X and Y. When the difference is reduced to a certain ratio, the subject will receive a feedback message, and then the subject can perform another brain wave pattern comparison. The brain waves collected by the local brain wave collection device 10 can also be converted into a new benchmark score of X′, and this new benchmark score of X′ will also be compared with the database, and a new goal of the neural feedback training is to decrease a difference between X′ and Y. The neural feedback training is very similar to muscle training of the brain. If you want to exercise a certain muscle (biceps of the hand, six-pack of the abdomen, thigh muscles of the legs, etc.), the muscle endurance before exercise is a strength of X, and the goal is to gain a strength of Y. At this time, you will gradually train specific muscle groups until a difference between X and Y gradually decreases. For example, if you want to lift a dumbbell with a weight of Y (30 kg), but the current strength of the user only supports X (10 kg), if the user can lift 15 kg, then it will give feedback (a difference between X and Y decreases with a certain ratio), until the exercise lasts for a period of time, and then evaluation is performed again, the user can lift dumbbells with a weight of X′ (20 kg), at this time, the user may need to lift a dumbbell with a weight of 25 kg (a difference between X′ and Y decreases by a certain percentage) to get feedback. For example, a user with inattention who wants to improve concentration through brain training may find that the frontal lobe region of the brain is over-activated compared with the norm by analysis of the collected brain waves, and then gradually reduce the difference between X and Y through feedback.

FIG. 6A illustrates a diagram of a local brain wave collection device according to an embodiment of the present invention. The four brain wave characteristics will be compared with the parameters of the four characteristics in the detection database 17, and evaluation and calculation of the brain wave (EEG) and heart rate variability (HRV) of the subject will be stored in the detection database 17, and the conversion device 18 is used to convert a difference between the brain wave characteristics of the subject and the detection database 17 into a standard score as a standard threshold value of a feedback device 19 (when the difference is greater, the degree of brain dysfunction is higher), the feedback device 19 will use visual and audial feedback through using a combination of brain wave locations and brain wave patterns of the 19 channels to build the active area of brain to be used as parameters of detection of the brain function and neurophysiological feedback training, as well as brain wave (EEG) pattern characteristics and heart rate variability (HRV) characteristics for feedback training. FIG. 6B is a side view of FIG. 6A, as shown in FIG. 6B, the local brain wave collection device is worn to contact the top of the subject's head, posterior occipital convexity, vestibule and the root of the nose. The local brain wave collection device includes electrodes CZ, PZ, OZ, FPZ, FZ, C3, P3, T5, T3, F7, F3 and the first connecting ear electrode A2.

The embodiment of the present invention trains the brain surface cortex through 19 channels of neurophysiological feedback, and also trains the deep brain cortex in depth, so that the stereo positioning is more accurate, and the specific brain regions can also be trained. Compare the change of the brain waves before and after training with that of the brain waves trained with conventional techniques, and only the electrode pads are used to disclose the different effects of neurophysiological feedback on sensorimotor rhythm.

The present invention includes real-time (within 1 minute) analysis and feedback, and the brain waves are greater than other physiological signals. In particular, the 19-channel “calculation” and “module comparison” are used to achieve wireless long-distance transmission and prevent the signal from distortion, and the present invention uses electroencephalogram (EEG) with high time resolution, and specific areas of the brain can be stereo-located through 19 channels.

In addition, although the dongle of the present invention is different from the existing dongle, the long-distance transmission of the existing dongle is usually only a signal output without signal input. The transmission of the dongle of the present invention can reach a distance more than 500 m and include three stages: signal transmission (user end), real-time calculation (cloud, supply end), signal feedback (user end). These three stages are immediate, accurate and free from interference from other noise. In addition to using a computer, the user of the platform of the present invention also can use functions include a screen synchronization control function achieved through authorization, so that professionals on the supply end in this case can use specially developed neural feedback software to adjust the training parameters of the subject. In addition, when a mobile phone is used for training, due to the limited memory capacity of the mobile phone, external computing software and hardware devices of the mobile phone is also used in some embodiments of the present invention, so that the subject can receive neurophysiological feedback training through the mobile phone.

By using the system and method of the present invention for biological feedback training, real time (within 1 minute) feedback can be provided remotely, so that the subject can immediately understand the condition, and the subject can adjust the physiological signals through visual or audial feedback for recovery, and the stereo-locating of neurophysiological feedback training is more accurate.

The present invention is not limited to the above-mentioned embodiments. It is obvious to those skilled in the art that various modifications and variations can be made to the present invention without departing from the spirit or scope of the present invention. Therefore, the present invention is intended to cover the modifications and variations made to the present invention or falling within the scope and the equivalent scope of claims.

What is claimed is:

1. A system for providing real-time biological feedback training through remote transmission comprising:
a local brain wave collection device for detecting a brain wave and a heart rate variability data of a subject to generate a biological database;
a dongle for establishing wireless or wired communication with the local brain wave collection device to receive the biological database; and
a docking device electrically connected to the dongle to upload the biological database to a remote cloud system for abnormal brain wave comparison, receive a feedback result from the remote cloud system, provide the feedback result through a feedback interface to the subject and allow the subject to adjust physiological signals for recovery;
wherein the remote cloud system includes a brain wave database, and the remote cloud system compares the biological database according to the brain wave database to generate the feedback result which is visual or aural in real time.

2. The system of claim 1, wherein the biological database is about brain wave data of 19 channels.

3. The system of claim 2, wherein the remote cloud system includes a cloud server which converts 19-channel brain waves into characteristics including vibration, frequency, brain wave location, and brain wave pattern through calculating frequency spectrum.

4. The system of claim 1, wherein the brain wave database includes health norm and clinical norm for comparing the biological database uploaded by the docking device.

5. The system of claim 4, wherein the remote cloud system includes a conversion device and a feedback device, the conversion device compares the biological database according to the brain wave database to generate a comparison result, and the feedback device generates a feedback result from the comparison result according to a training threshold.

6. The system of claim 5, wherein the feedback result based on locations of the 19 channels is used to build an active area of brain by a combination of brain wave patterns to train a specific area of the brain.

7. The system of claim 2, wherein the brain wave database includes health norm and clinical norm for comparing the biological database uploaded by the docking device.

8. The system of claim 7, wherein the remote cloud system includes a conversion device and a feedback device, the conversion device compares the biological database according to the brain wave database to generate a comparison result, and the feedback device generates a feedback result from the comparison result according to a training threshold.

9. The system of claim 8, wherein the feedback result based on locations of the 19 channels is used to build an active area of brain by a combination of brain wave patterns to train a specific area of the brain.

10. The system of claim 1, wherein the local brain wave collection device is a brain wave detection cap.

11. A method for providing real-time biological feedback training through remote transmission comprising:
using a local brain wave collection device to detect a brain wave and a heart rate variability data of a subject to generate a biological database;
using a dongle to establish wireless or wired communication with the local brain wave collection device to receive the biological database;
using a docking device electrically connected to the dongle to upload the biological database to a remote cloud system for abnormal brain wave comparison; and
receiving a feedback result from the remote cloud system to provide the feedback result through a feedback interface to the subject and allow the subject to adjust physiological signals for recovery according to the feedback result;
wherein the remote cloud system includes a brain wave database, and the remote cloud system compares the biological database according to the brain wave database to generate the feedback result which is visual or aural in real time.

12. The method of claim 11, wherein the biological database is about brain wave data of 19 channels.

13. The method of claim 12, wherein the remote cloud system includes a cloud server which converts 19-channel brain waves into characteristics including vibration, frequency, brain wave location, and brain wave pattern through calculating frequency spectrum.

14. The method of claim 11, wherein the brain wave database includes health norm and clinical norm for comparing the biological database uploaded by the docking device.

15. The method of claim 14, wherein the remote cloud system includes a conversion device and a feedback device, the conversion device compares the biological database according to the brain wave database to generate a comparison result, and the feedback device generates a feedback result from the comparison result according to a training threshold.

16. The method of claim 15, wherein the feedback result based on locations of the 19 channels is used to build an active area of brain by a combination of brain wave patterns to train a specific area of the brain.

17. The method of claim 12, wherein the brain wave database includes health norm and clinical norm for comparing the biological database uploaded by the docking device.

18. The method of claim 17, wherein the remote cloud system includes a conversion device and a feedback device, the conversion device compares the biological database according to the brain wave database to generate a comparison result, and the feedback device generates a feedback result from the comparison result according to a training threshold.

19. The method of claim 18, wherein the feedback result based on locations of the 19 channels is used to build an active area of brain by a combination of brain wave patterns to train a specific area of the brain.
